(19) United States
(12) Patent Application Publication
(10) Pub. No.: US 2024/0050041 A1
(43) Pub. Date: Feb. 15, 2024

(54) OPTICAL DEVICE

(71) Applicant: Leadtek Research Inc., New Taipei City (TW)

(72) Inventors: Ming-Rung Tsai, New Taipei City (TW); Yuan-Ta Shih, New Taipei City (TW)

(73) Assignee: Leadtek Research Inc., New Taipei City (TW)

(21) Appl. No.: 17/899,624

(22) Filed: Aug. 31, 2022

(30) Foreign Application Priority Data
Aug. 11, 2022 (TW) 111130200

(51) Int. Cl. A61B 5/00; G01N 21/31; G01N 33/483; G01N 33/49; A61B 5/0205; A61B 5/145; A61B 5/1455

(52) U.S. Cl. CPC A61B 5/7203; G01N 21/31; G01N 33/4833; G01N 33/49; A61B 5/0205; A61B 5/14546; A61B 5/1455; A61B 5/02416

(57) ABSTRACT

An optical device includes: a first substrate; a second substrate on the first substrate; multiple optical sensing modules on the first substrate, the multiple optical sensing modules including a first group of optical sensing modules and a second group of optical sensing modules; and a controller electrically connected to the multiple optical sensing modules. When the controller detects a user, the controller sends control signals to drive the first group of optical sensing modules and the second group of optical sensing modules simultaneously to perform a measurement on the user, and obtains a first physiological signal of the user and a second physiological signal of the user simultaneously.

### CROSS-REFERENCE TO RELATED APPLICATION

This application claims the priority benefit of Taiwan application serial no. 111130200, filed on Aug. 11, 2022. The entirety of the above-mentioned patent application is hereby incorporated by reference herein and made a part of this specification.

### BACKGROUND

### Technical Field

The disclosure relates to an optical device.

### Description of Related Art

Optical measurement technology is often used to non-invasively detect the type or content of different substances in biological tissues, so as to provide characteristics of biological tissues as a reference for medical diagnosis or home monitoring of physical health index. With the popularization and diversity of light emitting diode light sources, the use of optical devices to measure physiological signals has the advantages of portability and low cost. However, optical measurement is easily affected by the heterogeneity of biological tissues, which in turn affects the accuracy of measurement results. Generally speaking, in order to improve the accuracy of measurement results, the number of light sources (emitters) or light detectors (detectors) is increased. In addition, due to the different optical principles and mechanisms for measuring different biological tissue parameters, it is usually necessary to measure them separately. In addition to increasing the measurement time, it also increases the risk of instability.

### SUMMARY

The disclosure provides a multi-channel optical device to reduce the measurement result error caused by the heterogeneity of biological tissue, and may reduce the measurement time, thereby improving the measurement accuracy and stability.

An optical device includes: a first substrate; a second substrate on the first substrate; multiple optical sensing modules on the first substrate, the multiple optical sensing modules including a first group of optical sensing modules and a second group of optical sensing modules; and a controller electrically connected to the multiple optical sensing modules. When the controller detects a user, the controller simultaneously sends control signals and drives the first group of optical sensing modules and the second group of optical sensing modules to perform a measurement on the user, and obtains a first physiological signal of the user and a second physiological signal of the user simultaneously.

Based on the above, the optical device of the disclosure may simultaneously measure multiple physiological signals of the user, and provide at least two different measurement areas through at least two channels to reduce the measurement error caused by the heterogeneity of biological tissues, thereby increasing the accuracy of the measurement, which may greatly reduce the measurement time and manpower. In addition, the optical device of the disclosure may measure two different biological tissue parameters at the same time, which may greatly reduce the measurement time, thereby improving the stability of the measurement.

### BRIEF DESCRIPTION OF THE DRAWINGS

FIG. 1 is a schematic diagram of an optical device according to an embodiment of the disclosure.

FIG. 2 is a schematic block diagram of an optical device according to an embodiment of the disclosure.

FIG. 3 is an electronic signal diagram of an optical device according to an embodiment of the disclosure.

### DESCRIPTION OF EMBODIMENTS

The following embodiments are described in detail in conjunction with the accompanying drawings, but the provided embodiments are not intended to limit the scope of the disclosure. In addition, the dimensions of the components in the drawings are drawn for the convenience of description, and do not represent the actual proportions of the component dimensions. To facilitate understanding, hereinafter, similar elements will be denoted by the same reference numerals.

Different examples in the description of the embodiments of the disclosure may use repeated reference numerals and/or terms. These repeated reference numerals or terms are for the purpose of simplification and clarity, and are not used to limit the relationship of each embodiment and/or the structure. Furthermore, if the following description describes that a first feature is formed on or above a second feature, it means to include embodiments in which the first feature and the second feature are formed in direct contact, and also to include embodiments in which an additional feature is formed between the first and second features, such that the first and second features may not be in direct contact.

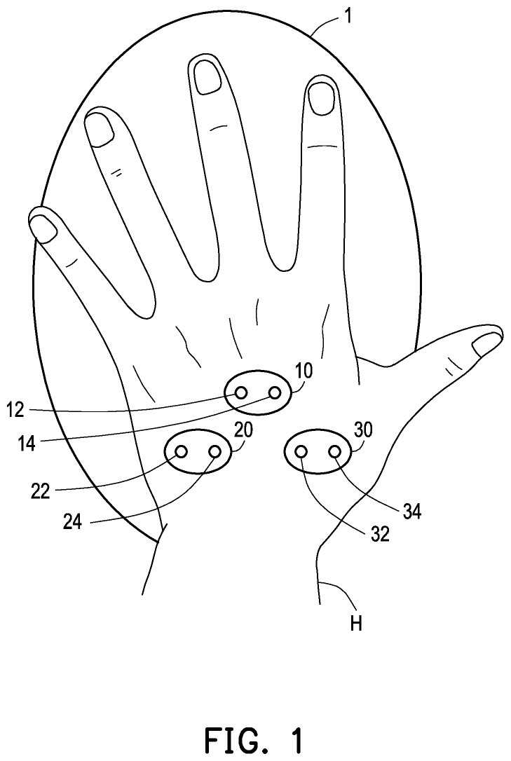

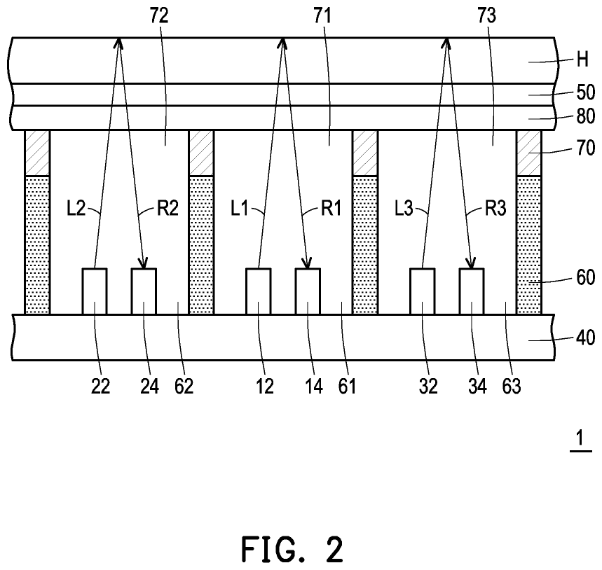

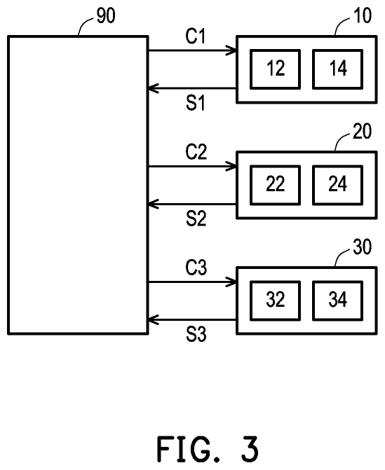

FIG. 1 is a schematic diagram of an optical device according to an embodiment of the disclosure. FIG. 2 is a schematic block diagram of an optical device according to an embodiment of the disclosure. FIG. 3 is an electronic signal diagram of an optical device according to an embodiment of the disclosure.

Please refer to FIG. 1. An optical device 1 has optical sensing modules 10, 20 and 30 distributed in different positions of the optical device 1. A part of a user H is placed on the optical device 1. The optical sensing modules 10, 20 and 30 are configured to measure multiple physiological signals at different positions of the user H.

In this embodiment, the user H places his or her left hand on the optical device 1, and touches the optical device 1 with his or her palm. In other embodiments, the user H may contact the optical device 1 with other parts of the body, such as the right hand or other parts, which is not limited in the disclosure.

Please refer to FIG. 1, FIG. 2, and FIG. 3 at the same time. The optical device 1 includes a first substrate 40, a second substrate 50, multiple optical sensing modules 10, 20 and 30, and a controller 90.

The second substrate 50 is located on the first substrate 40. According to some embodiments, the material of the second substrate 50 is a light-transmitting material, such as glass or plastic material, but the disclosure is not limited thereto.

The optical sensing modules 10, 20, and 30 are located on the first substrate 40. According to some embodiments, the number of optical sensing modules is two or more. For example, in this embodiment, the number of optical sensing modules is three, which include the optical sensing modules 10, 20 and 30. The upper limit of the number of optical sensing modules is determined according to the volume size and usage requirements of the optical device 1, such as the number of physiological signals to be measured, and the disclosure is not limited thereto.

As shown in FIGS. 1 and 3, each of the optical sensing modules 10, 20, and 30 includes at least one light source and at least one light detector. For example, the optical sensing module includes a light source 12 and a light detector 14; the optical sensing module 20 includes a light source 22 and a light detector 24; and the optical sensing module 30 includes a light source 32 and a light detector 34. According to some embodiments, in the optical sensing module, the number of light sources and the number of light detectors may be determined according to actual requirements, which is not limited by the disclosure.

The light sources 12, 22, and 32 may emit color light beams L1, L2, and L3 for illuminating the user H. According to some embodiments, the light sources 12, 22 and 32 may be light emitting diodes, or other optical elements capable of emitting monochromatic light, but the disclosure is not limited thereto. The wavelength range of the color light beams L1, L2, and L3 is determined by the physiological signal to be measured, and the disclosure is not limited thereto. According to some embodiments, the light sources 12, 22, and 32 may emit light beams of different colors or the same colors; for example, the light source 12 may emit a first color light beam L1, and the light sources 22 and 32 may emit second color light beams L2 and L3, and the wavelength of the first color light beam L1 is different from the wavelength of the second color light beams L2 and L3. According to some embodiments, the first color light beam L1 may be red light or green light, and the second color light beams L2 and L3 may be blue light, but the disclosure is not limited thereto.

The light detectors 14, 24, and 34 are configured to receive reflected light beams R1, R2, and R3 reflected by the user H. According to some embodiments, the light detectors 14, 24, and 34 may include, for example, a charge coupled device image sensor (CCD image sensor) or a complementary metal oxide semiconductor (CMOS) or other similar elements, and the disclosure is not limited thereto.

According to some embodiments, the number of optical sensing modules emitting the first color light beam L1 and the wavelength of the first color light beam L1 and the number of optical sensing modules emitting the second color light beam L2 and the wavelength of the second color light beam L2 may be determined according to actual requirements, such as the physiological signal to be detected, which is not limited in the disclosure.

As shown in FIG. 2, the controller 90 is electrically connected to the multiple optical sensing modules 10, 20, and 30. The controller 90 sends control signals C1, C2, and C3 to drive the optical sensing modules 10, 20, and 30, respectively. The controller sends the control signals C1, C2, and C3 to control the lighting states of the light sources 12, 22, and 32, such as emitting color light beams L1, L2, and L3 or stopping emitting color light beams L1, L2, and L3. The control signals C1, C2, and C3 of the controller 90 also control the states of the light detectors 14, 24, and 34 at the same time, receive the reflected light beams R1, R2, and R3 reflected by the user H, and convert the reflected light beams R1, R2, and R3 into electrical signals S1, S2, and S3, respectively, and the electrical signals S1, S2, and S3 are returned to the controller 90.

In other words, the controller 90 may drive the optical sensing modules 10, 20, and 30 through the control signals C1, C2, and C3, respectively and independently. According to some embodiments, the controller 90 may be a microprocessor, or a device having similar elements, and the disclosure is not limited thereto.

As shown in FIGS. 1 and 2, the optical device 1 further includes an isolation structure 60 disposed between the first substrate 40 and the second substrate 50. According to some embodiments, the material of the isolation structure 60 is a light-absorbing material, a metal material with high reflectivity, or a non-metallic material with high reflectivity, such as black resin, white resin, or other suitable light-absorbing or reflective materials, but the disclosure is not limited thereto.

The isolation structure 60 includes multiple through holes 61, 62, and 63. In this embodiment, the number of the through holes 61, 62 and 63 is equal to the number of the optical sensing modules 10, 20 and 30, and each of the optical sensing modules 10, 20 and 30 corresponds to each of the through holes 61, 62, and 63, respectively, and is located in each of the multiple through holes 61, 62, and 63. Therefore, the color light beams L1, L2, and L3 emitted by the optical sensing modules 10, 20, and 30 and the reflected light beams R1, R2, and R3 reflected by the user H may be limited to the through holes 61, 62, and 63 corresponding to each of the optical sensing modules 10, 20, and 30, which may avoid the situation of mutual interference and increase the accuracy of measuring the physiological signals of the user.

As shown in FIG. 2, the optical device 1 further includes an aperture layer 70. The aperture layer 70 is located between the multiple optical sensing modules 10, 20, and 30 and the second substrate 50, and is disposed on the isolation structure 60. According to some embodiments, the material of the aperture layer 70 is a light-absorbing material, a metal material with high reflectivity, or a non-metallic material with high reflectivity, such as black resin, white resin, or other suitable light-absorbing or reflective materials, but the disclosure is not limited thereto.

The aperture layer 70 includes multiple apertures 71, 72, and 73. The number of the multiple apertures 71, 72, and 73 is equal to the number of the multiple optical sensing modules 10, 20, and 30. The positions of the multiple apertures 71, 72 and 73 each correspond to the positions of the multiple through holes 61, 62 and 63, and also correspond to the positions of the multiple optical sensing modules 10, 20 and 30. The aperture layer 70 is configured to isolate the background interference light from being incident on the through holes 71, 72, and 73 and the optical sensing modules 10, 20 and 30. The apertures 71, 72, and 73 are configured to allow the color light beams L1, L2, and L3 and the reflected light beams R1, R2, and R3 to pass through.

In some embodiments, as shown in FIG. 2, the optical device 1 may include a lens layer 80. In other embodiments, the optical device 1 may not include the lens layer 80. The lens layer 80 is located between the multiple optical sensing modules 10, 20, and 30 and the second substrate 50, and is disposed on the aperture layer 70. According to some embodiments, the lens layer 80 has multiple microlenses, and the material of the lens layer includes glass or plastic, but is not limited thereto. The lens layer 80 is configured to focus the color light beams L1, L2, and L3 and the reflected light beams R1, R2, and R3 emitted by the optical sensing modules 10, 20, and 30 to increase the accuracy of measuring physiological signals.

According to some embodiments, whether to provide the isolation structure 60, the aperture layer 70 and the lens layer 80 in the optical device 1 may be determined according to actual requirements. In some embodiments, the optical device 1 may not include the isolation structure 60, the aperture layer 70 and the lens layer 80. In other embodiments, the optical device 1 may include some or all of the isolation structure 60, the aperture layer 70 and the lens layer 80, but the disclosure is not limited thereto. If the distance between the optical sensing modules 10, 20, and 30 is far enough, or the distance between the optical sensing modules 10, 20, and 30 and the user H is close enough to prevent the color light beams emitted by each optical sensing module and the reflected light beams from interfering with each other, it may be considered to provide only the isolation structure 60 or only the aperture layer 70. If the color light beams L1, L2, and L3 emitted by the optical sensing modules 10, 20, and 30 are sufficiently concentrated, it may be considered not to dispose the lens layer 80.

The following describes a method for measuring multiple physiological signals of the user H with the optical device 1.

Please refer to FIG. 1, FIG. 2, and FIG. 3 at the same time. The optical device 1 has multiple optical sensing modules, that is, the optical sensing modules 10, 20 and 30. According to some embodiments, the multiple optical sensing modules may have multiple groups of multiple optical sensing modules, and each group of multiple optical sensing modules corresponds to measuring a physiological signal of the user. In the disclosure, the number of the optical sensing module is two or more. In this embodiment, the multiple optical sensing modules include a first plurality of optical sensing modules for measuring a first physiological signal of the user and a second plurality of optical sensing modules for measuring a second physiological signal of the user. In some embodiments, the controller 90 may drive only the corresponding plurality of optical sensing modules according to the physiological signal to be measured. For example, in some embodiments, if only the first physiological signal of the user is to be measured, the controller sends a control signal to drive the first plurality of optical sensing modules for measuring the first physiological signal of the user, and do not drive the multiple optical sensing modules for measuring other physiological signals of the user; for example, the controller does not drive the second plurality of optical sensing modules for measuring the second physiological signal of the user.

According to some embodiments, the number of the first plurality of optical sensing modules is one or more. According to some embodiments, the number of the second plurality of optical sensing modules is one or more. Therefore, the total number of optical sensing modules is at least two, that is, the first plurality of optical sensing modules include one optical sensing module, and the second plurality of optical sensing modules include one optical sensing module. In this embodiment, as shown in FIG. 1 and FIG. 3, the first plurality of optical sensing modules include the optical sensing module 10, and the second plurality of optical sensing modules include the optical sensing modules 20 and 30.

Please refer to FIG. 3. When the controller 90 detects the user H, for example, when the controller 90 detects that the user H contacts the second substrate 50, the controller 90 sends out the control signals C1, C2, and C3 to simultaneously drive the first plurality of optical sensing modules (that is, the optical sensing module 10) and the second plurality of optical sensing modules (that is, the optical sensing modules 20 and 30) to simultaneously obtain the first physiological signal of the user H and the second physiological signal of the user H.

According to some embodiments, the physiological signals referred to in the disclosure include: heart rate, blood pressure, blood oxygen value, blood glucose value, carotenoid value, and the like, but are not limited thereto.

Specifically, when the controller 90 detects the user H, for example, when the controller 90 detects that the user H contacts the second substrate 50, the controller 90 sends out the control signals C1, C2, and C3 to simultaneously drive the light source 12 of each optical sensing module of the first plurality of optical sensing modules to emit the first color light beam L1, and the controller 90 drives the light sources 22 and 32 of each optical sensing module 20 and 30 of the second plurality of optical sensing modules to emit the second color light beams L2 and L3. The first color light beam L1 and the second color light beams L2 and L3 are incident on the contact part between the user H and the second substrate through the through holes 61, 62, and 63, the apertures 71, 72, and 73, the lens layer 80 and the second substrate 50, and are reflected by the contact part between the user H and the second substrate. In this embodiment, the contact part between the user H and the second substrate is the palm of the user, but it is not limited thereto.

The first reflected light beam R1 reflected by the user H is received by the light detector 14 of each optical sensing module 10 of the first plurality of optical sensing modules, and the light detector 14 converts the first reflected light beam R1 into the first electrical signal S1 and transmits it to the controller 90. The second reflected light beams R2 and R3 reflected by the user H are received by the light detectors 24 and 34 of each optical sensing module 20 and 30 of the second plurality of optical sensing modules, and the light detectors 24 and 34 convert the second reflected light beams R2 and R3 into the second electrical signals S2 and S3 and transmit them to the controller 90. The controller 90 obtains the first physiological signal of the user H according to the first electrical signal S1, and the controller 90 obtains the second physiological signal of the user H according to the second electrical signals S2 and S3.

When the first color light beam L1 and the second color light beams L2 and L3 are incident on the user H, because the tissue structure of each part of the user H is slightly different, such as the structural differences in skin and muscle thickness, blood vessel distribution and the like, the user H has different absorptivity for the first color light beam L1 and the second color light beams L2 and L3, thereby changing the intensities of the reflected light beams R1, R2 and

R3 reflected by the user H. Therefore, when the number of a certain group of optical sensing modules configured to measure a certain physiological signal is two or more, the results measured by this group of optical sensing modules may be averaged to obtain a more accurate physiological signal. In this embodiment, the number of the optical sensing modules of the second plurality of optical sensing modules 20 and 30 is two or more. At this time, the controller 90 calculates the corresponding second physiological signal for each of the second electrical signals S2 and S3 of the second plurality of optical sensing modules 20 and 30 respectively, and averages the physiological signals measured by the optical sensing modules 20 and 30 to obtain the second physiological signal. By using multiple optical sensing modules for measurement and averaging, the error of the measurement result of the physiological signal caused by the heterogeneity of the biological tissues of the user may be effectively reduced.

As shown in FIG. 1 to FIG. 3, in an embodiment, the first physiological signal is a heart rate, and the second physiological signal is a carotenoid value. The first plurality of optical sensing modules include the optical sensing module 10, and the second plurality of optical sensing modules include the optical sensing modules 20 and 30. The light source 12 of the optical sensing module 10 emits the first color light beam L1, which is red light. The light sources 22 and 32 of the optical sensing modules 20 and 30 emit the second color light beams L2 and L3, which are blue light. When the first color light beam L1 is irradiated on the user H, the first color light beam L1, that is, red light, is absorbed by heme in the blood vessel. By measuring the changes in the intensity of the reflected light beam R1 with time, the heart rate value of the user H may be calculated. When the second color light beams L2 and L3 are irradiated on the user H, the second color light beams L2 and L3, that is, blue light, are absorbed by carotenoids in the skin and blood vessels. By measuring the changes in the intensity of the reflected light beams R2 and R3 with time, the carotenoid values in the skin and blood of the user H may be calculated.

The first reflected light beam R1 reflected by the user H is received by the light detector 14 of the optical sensing module 10 of the first plurality of optical sensing modules, and the light detector 14 converts the first reflected light beam R1 into the first electrical signal S1 and transmits it to the controller 90. The controller 90 obtains the first physiological signal of the user, that is, the heart rate, according to the first electrical signal S1.

The second reflected light beams R2 and R3 reflected by the user H are received by the light detectors 24 and 34 of the optical sensing modules 20 and 30 of the second plurality of optical sensing modules, and the light detector 24 and 34 convert the second reflected light beams R2 and R3 into the second electrical signals S2 and S3 and transmit them to the controller 90. Since the number of the optical sensing modules 20 and 30 of the second plurality of optical sensing modules is two or more, the controller 90 calculates the corresponding second physiological signals for each of the second electrical signals S2 and S3 of the optical sensing modules 20 and 30 respectively, and averages the physiological signals measured by the optical sensing modules 20 and 30 to obtain the second physiological signal, that is, the carotenoid value in the skin and blood. By averaging the physiological signals measured by the optical sensing modules 20 and 30, the error in the measurement results of the physiological signals caused by the heterogeneity of the biological tissues of the user may be effectively reduced.

According to the embodiments described in the disclosure, the optical device of the disclosure may simultaneously measure two or more physiological signals, thereby effectively reducing the measurement time. When two or more optical sensing modules are used to measure the same physiological signal, the physiological signals obtained by the two or more optical sensing modules may be averaged to obtain the averaged physiological signal, which may effectively reduce the error in the measurement result of the physiological signal caused by the heterogeneity of the biological tissues of the user.

Although the disclosure has been described above with the embodiments, the embodiments are not intended to limit the disclosure. One with ordinary skill in the art may make some changes and modifications without departing from the spirit and scope of the disclosure. Therefore, the protection scope of the disclosure shall be determined by the scope of the appended claims and the equivalents thereof

What is claimed is:

1. An optical device comprising:
a first substrate;
a second substrate located on the first substrate;
a plurality of optical sensing modules located on the first substrate; and
a controller electrically connected to the plurality of optical sensing modules,
wherein in response to detecting a user, the controller sends a control signal to drive a first plurality of the optical sensing modules and a second plurality of the optical sensing modules simultaneously to perform a measurement on the user, and obtains a first physiological signal of the user and a second physiological signal of the user simultaneously.

2. The optical device according to claim 1, wherein each of the plurality of optical sensing modules comprises at least one light source and at least one light detector, and the light source and the light detector are electrically connected to the controller,
wherein in response to detecting the user,
the controller sends a control signal to drive the light source of each of the first plurality of optical sensing modules to emit a first color light beam, the light detector of each of the first plurality of optical sensing modules receives a first reflected light beam reflected by the user, and the light detector converts the first reflected light beam into a first electrical signal and transmits the first electrical signal to the controller,
the controller drives the light source of each of the second plurality of optical sensing modules to emit a second color light beam, the light detector of each of the second plurality of optical sensing modules receives a second reflected light beam reflected by the user, and the light detector converts the second reflected light beam into a second electrical signal and transmits the second electrical signal to the controller, and
the controller obtains the first physiological signal of the user according to the first electrical signal, and the controller obtains the second physiological signal of the user according to the second electrical signal,
wherein the controller simultaneously drives the light source of each of the first plurality of optical sensing modules to emit the first color light beam and the light source of each of the second plurality of optical sensing modules to emit the second color light beam, respectively.

3. The optical device according to claim 2, wherein a wavelength of the first color light beam is different from a wavelength of the second color light beam.

4. The optical device according to claim 2, wherein the first color light beam is red light or green light.

5. The optical device according to claim 2, wherein the second color light beam is blue light.

6. The optical device according to claim 1, wherein the number of the first plurality of optical sensing modules is one or more.

7. The optical device according to claim 1, wherein the number of the second plurality of optical sensing modules is one or more.

8. The optical device according to claim 2, wherein the number of the second plurality of optical sensing modules is two or more, and the controller calculates a corresponding physiological signal for each of the second electrical signals of the second plurality of optical sensing modules and averages the physiological signals to obtain the second physiological signal.

9. The optical device according to claim 1, wherein the first physiological signal comprises a heart rate.

10. The optical device according to claim 1, wherein the second physiological signal comprises a carotenoid value.

11. The optical device according to claim 1, wherein the controller is a microprocessor.

12. The optical device according to claim 1, further comprising:
an isolation structure comprising a plurality of through holes, wherein the isolation structure is disposed between the first substrate and the second substrate, the number of the plurality of through holes is equal to the number of the plurality of optical sensing modules, each of the plurality of optical sensing modules corresponds to each of the plurality of through holes, and is located in each of the plurality of through holes.

13. The optical device according to claim 1, further comprising:
an aperture layer comprising a plurality of apertures, wherein the aperture layer is disposed between the plurality of optical sensing modules and the second substrate, and the number of the plurality of apertures is equal to the number of the plurality of optical sensing modules, and a position of each of the plurality of apertures corresponds to a position of the plurality of optical sensing modules.

14. The optical device according to claim 1, further comprising:
a lens layer configured to focus a plurality of color light beams emitted by the plurality of optical sensing modules, wherein the lens layer is disposed between the plurality of optical sensing modules and the second substrate.
